(19) United States
(12) Patent Application Publication
(10) Pub. No.: US 2014/0342442 A1
(43) Pub. Date: Nov. 20, 2014

(54) TOUCHSCREEN DEVICE AND METHODS FOR USE IN DETECTION OF MICRORNA

(71) Applicant: BIOO SCIENTIFIC CORPORATION, Austin, TX (US)

(72) Inventors: Masoud M. Toloue, Austin, TX (US); Michael T. Mueller, Jarrell, TX (US); Swapna Gone, Austin, TX (US)

(73) Assignee: BIOO SCIENTIFIC CORPORATION, Austin, TX (US)

(21) Appl. No.: 14/278,996

(22) Filed: May 15, 2014

Related U.S. Application Data
(60) Provisional application No. 61/823,617, filed on May 15, 2013.

(51) Int. Cl. C12Q 1/68; G06F 3/044

(52) U.S. Cl. CPC C12Q 1/6834; G06F 3/044; G06F 2203/04104
USPC 435/287.2

(57) ABSTRACT

A method and sensor for detecting the presence of specific target nucleic acids uses a biosensor comprising an array of single stranded probe oligonucleotides, wherein one end of each probe oligonucleotide is linked to a label and the other end of each probe oligonucleotide is attached to an electrode of a solid support. The method includes determining the capacitance at one or more of the electrodes, where a change in the capacitance of the one or more electrodes indicates the presence of target nucleic acid complementary to the single stranded oligonucleotide probe.

### PRIORITY CLAIM

This application claims priority to U.S. Provisional Application Ser. No. 61/823,617 entitled “TOUCHSCREEN DEVICE AND METHODS FOR USE IN DETECTION OF MICRORNA” filed May 15, 2013, which is incorporated herein by reference in its entirety.

### STATEMENT REGARDING FEDERALLY SPONSORED RESEARCH OR DEVELOPMENT

This invention was made with government support under NSF Phase II grant no. 1230440 and NSF Phase I grant no. 1047285. The government has certain rights in the invention.

### BACKGROUND OF THE INVENTION

1. Field of the Invention

The invention generally relates to biosensors. More particularly the invention relates to biosensors and methods for detecting the presence of specific target nucleic acids in a sample.

2. Description of the Relevant Art

miRNAs are small 21-23 base oligonucleotides that are involved in regulation of gene expression. There are approximately 1,000 different miRNAs in humans. Differences in miRNA expression (e.g. up-regulation, down-regulation) have been implicated in disease states (e.g. cancer, tumorigenesis), and thus offer potential for use in life sciences research or as a diagnostic.

There is a desire for quantitative, multiplexed, global gene expression profiling capable of identifying differences in expression of all microRNAs in all organisms. Furthermore, there is a desire for readout systems that do not require expensive or bulky instrumentation, such as optical or fluorescent plate readers.

Furthermore, there is a desire for miRNA panels that can be used for diagnosis of disease, for life sciences research, for basic research, and for screening of pharmaceutical, pharmacologic, or biologic compounds.

### SUMMARY OF THE INVENTION

In an embodiment, a method for detecting the presence of specific target nucleic acids in a sample, includes: obtaining a biosensor comprising an array of single stranded probe oligonucleotides, wherein one end of each probe oligonucleotide is linked to a label and the other end of each probe oligonucleotide is coupled to an electrode on a solid support; contacting the probes arrayed on the biosensor with the sample; storing the sample and biosensor under conditions effective to allow hybridization of the probes to the target nucleic acids, if present, wherein said hybridization converts the probes from single stranded to double stranded; treating the biosensor with a reagent effective to selectively remove single stranded probes along with the associated label; and determining the post-treatment capacitance at one or more of the electrodes, where a change in the capacitance of the one or more electrodes, with respect to the capacitance measured at an electrode indicates the presence of target nucleic acid complementary to the single stranded oligonucleotide probe.

In one embodiment, the biosensor is treated with a cleavage enzyme that is specific for cleaving single stranded oligonucleotides. Exemplary cleavage enzymes include, but are not limited to S1 endonuclease and RNase III.

The method for detecting the presence of specific target nucleic acids in a sample can be used for a variety of purposes. For example, the specific target nucleic acids may be: cancer diagnostic nucleic acids; gene expression profiling nucleic acids; gene up-regulation nucleic acids; gene down-regulation nucleic acids; or wherein the specific target nucleic acids are produced in response to a pharmaceutical compound.

In an embodiment, a biosensor includes: a substrate comprising a plurality of capacitive sensitive electrodes; one or more single stranded probe oligonucleotides, wherein one end of each single stranded probe oligonucleotide is linked to a label and the other end of each single stranded probe oligonucleotide is coupled to the substrate proximate to one or the plurality of electrodes; wherein the capacitance of the electrodes changes when the coupled single stranded probe oligonucleotide is removed or altered.

The substrate, in an embodiment, may be a touchscreen. For example, the substrate may be a capacitive touchscreen, more particularly, a projected capacitive touchscreen. The substrate may include a glass layer formed over one or more of the electrodes. The one or more single stranded probe oligonucleotides may be coupled to the glass layer. In one embodiment, the substrate includes glass having an electrode array formed of indium tin oxide on a surface of the glass. The substrate may be incorporated into a cell phone or a computer.

In an embodiment, the one or more of the single stranded probe oligonucleotides are: DNA oligonucleotides; oligonucleotides capable of hybridizing with miRNA; or oligonucleotides capable of hybridizing with cDNA. One or more of the single stranded probe oligonucleotides have a magnetic bead attached to the 5′ end, wherein the magnetic bead is the label. One or more of the single stranded probe oligonucleotides have a magnetic bead attached to the 3′ end, wherein the magnetic bead is the label. One or more of the single stranded probe oligonucleotides are coupled to the substrate at the 3′ end. One or more of the single stranded probe oligonucleotides are coupled to the substrate at the 5′ end.

In an embodiment, a method of making a biosensor includes: obtaining a substrate comprising a plurality of capacitive sensitive electrodes; and coupling a plurality of single stranded probe oligonucleotides to the substrate, wherein one end of each single stranded probe oligonucleotide is linked to a label and the other end of each single stranded probe oligonucleotide is coupled to the substrate proximate to one or the plurality of electrodes. The oligonucleotides may be coupled to the substrate using microarray spotting or inkjet printing.

In an alternate embodiment, a biosensor includes: a substrate comprising one or more electrodes on a first side of the substrate and a grounding layer on a second side of the substrate, the second side being opposite to the first side; one or more single stranded probe oligonucleotides, wherein one end of each single stranded probe oligonucleotide is linked to a label and the other end of each single stranded probe oligonucleotide is coupled to the electrodes of the first substrate; and vias connecting the one or more electrodes on the first side of the first substrate to the grounding layer on the second side of the second substrate. The capacitance of the touchscreen sensor changes when the first substrate containing coupled single stranded probe oligonucleotide contained on the first substrate and in contact with the touchscreen sensor, is removed or altered.

In one embodiment, the grounding layer of the first substrate is placed in electrical contact with a ground. The ground may be a system ground. The ground may be the system ground of the capacitive touchscreen sensor. In an embodiment, the substrate is a printed circuit board or an integrated circuit.

In an embodiment, a method for detecting the presence of specific target nucleic acids in a sample, includes: obtaining a biosensor as described above; contacting the probes arrayed on the biosensor with the sample; storing the sample and biosensor under conditions effective to allow hybridization of the probes to the target nucleic acids, if present, wherein said hybridization converts the probes from single stranded to double stranded; treating the biosensor with a reagent effective to selectively remove single stranded probes along with the associated label; and placing the biosensor in contact with a capacitive touchscreen device, wherein the capacitive touchscreen device is capable of measuring capacitive changes due to contact of the biosensor with the capacitive touchscreen device.

### BRIEF DESCRIPTION OF THE DRAWINGS

Advantages of the present invention will become apparent to those skilled in the art with the benefit of the following detailed description of embodiments and upon reference to the accompanying drawings in which:

FIG. 1 depicts a schematic diagram of a method of using a biosensor having single strand oligonucleotide probes;

FIG. 2 depicts a second embodiment for performing an array of measurements of biological samples using a biosensor having an array or electrodes;

FIG. 3 depicts an experimental set up that includes a projected capacitive touchscreen coupled to a touchscreen controller;

FIG. 4 depicts a graph showing the change in capacitance due to the presence of water droplets on a projected capacitive touchscreen;

FIG. 5 depicts a graph showing the change in capacitance over time for water droplets with and without magnetic beads;

FIG. 6A depicts an experimental set up for determining the location of magnetic beads in water droplets disposed on a projected capacitive touchscreen;

FIG. 6B depicts a graph generated after signal processing of the experimental set up of FIG. 6A; and

FIG. 7 depicts a fabrication process for making a biosensor device.

While the invention may be susceptible to various modifications and alternative forms, specific embodiments thereof are shown by way of example in the drawings and will herein be described in detail. The drawings may not be to scale. It should be understood, however, that the drawings and detailed description thereto are not intended to limit the invention to the particular form disclosed, but to the contrary, the intention is to cover all modifications, equivalents, and alternatives falling within the spirit and scope of the present invention as defined by the appended claims.

### DETAILED DESCRIPTION OF THE PREFERRED EMBODIMENTS

It is to be understood the present invention is not limited to particular devices or methods, which may, of course, vary. It is also to be understood that the terminology used herein is for the purpose of describing particular embodiments only, and is not intended to be limiting. As used in this specification and the appended claims, the singular forms “a”, “an”, and “the” include singular and plural referents unless the content clearly dictates otherwise. Furthermore, the word “may” is used throughout this application in a permissive sense (i.e., having the potential to, being able to), not in a mandatory sense (i.e., must). The term “include,” and derivations thereof, mean “including, but not limited to.” The term “coupled” means directly or indirectly connected.

In an embodiment, a biosensor uses a substrate that includes a plurality of capacitive sensitive electrodes to non-optically (e.g., electronically) detect nucleic acids in a sample. For example, such a system may be used to: determine changes in miRNA expression. The use of an array of electrodes allows the biosensor to be both multiplexed and quantitative.

The biosensor should not be seen as being limited to the use of oligonucleotide probes. One skilled in the art will recognize and appreciate that other biomolecules may be utilized as probes. Other probes that may be used include, but are not limited to, proteins, hormones, neurotransmitters, small molecules, pharmaceutical compounds, phage, and bacteria.

In an embodiment, the biosensor may be composed of an array of probes. Hundreds to thousands or more probes can be deposited onto the substrate of the biosensor using microarray spotting, inkjet printing, spin-coating, or other suitable deposition means. In an embodiment, an array of single stranded probe oligonucleotides is used in the biosensor. One end of each single stranded probe oligonucleotide is linked to a label and the other end of each single stranded probe oligonucleotide is attached to a solid support.

In an embodiment, an oligonucleotide probe with an attached label (for example, a ferromagnetic label, a magnetic bead, a nanoparticle, or other suitable label) is deposited onto the sensor surface. A “label”, as used herein, is any compound capable of producing a sufficient change in measured capacitance in the presence of the substrate electrodes. For example, silver particles, which have high thermal and electrical conductivity, or graphene, which is a high dielectric material, may serve as suitable labels. In one embodiment, magnetic beads may be used since there are well-established protocols for attaching molecular biology materials such as nucleotides to such labels.

If the probe or target produces a sufficiently large change in capacitance, the use of a label may not be required at all, thus a direct label-free measurement using the biosensor substrate may be utilized.

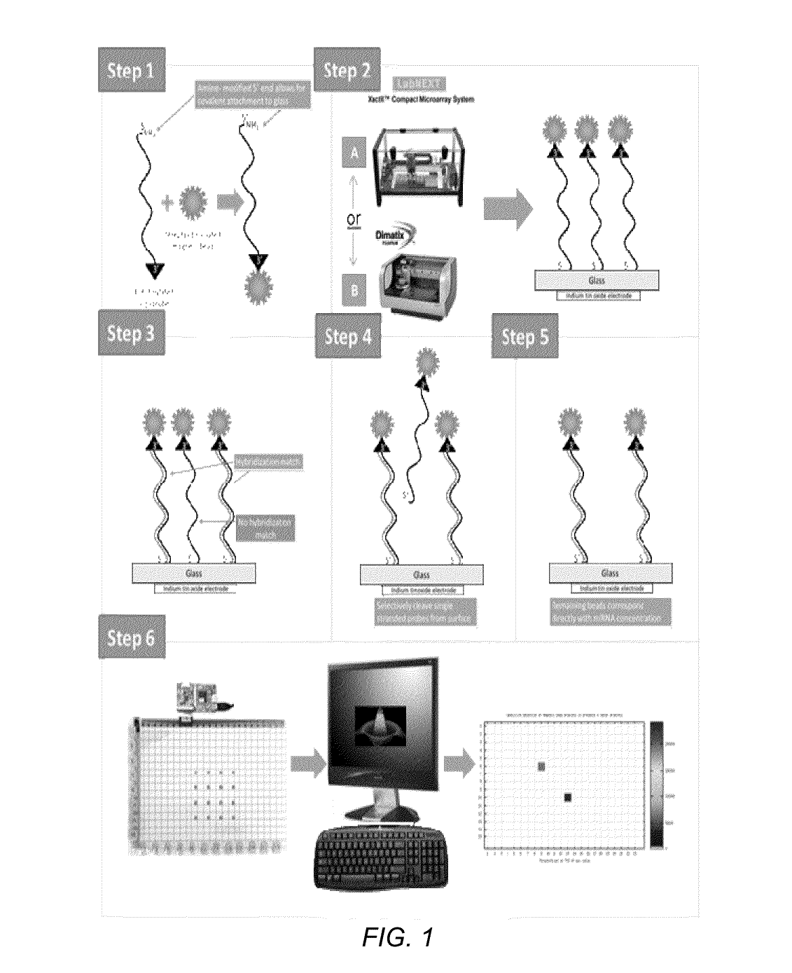

A schematic diagram of a method of using a biosensor having single strand oligonucleotide probes is depicted in FIG. 1. In step 1, single strand oligonucleotide probes are formed having a labeled (e.g., a magnetic bead) 3′ end, leaving the 5′ end available for coupling to the substrate. In step 2, the single strand oligonucleotide probes are coupled to a substrate having one or more electrodes. When using oligonucleotide probes, if the target sample is complementary to the base pair sequence, the target is hybridized to the probe (as shown in step 3). In step 4, a cleavage enzyme, such as an S1 nuclease or RNase III, is then used to selectively cleave single-stranded DNA (e.g. unhybridized probes) or ds-RNA (formed from immobilized RNA probe hybridizing to miRNA target). A wash (step 5) is performed to remove the cleaved single-stranded DNA including the attached label. The displacement of the label locally alters the electric and magnetic field in the vicinity of the electrodes of the substrate. Prior to use a baseline measurement of the capacitance of the electrodes is made in the absence of the labeled probe. After the labeled probes are attached, the capacitance of the electrodes is measured. During use of the biosensor, the capacitance of each electrode is modified as a result of the changing number of probes having labels in the vicinity of the electrodes. The difference in the signal may be correlated to the amount of the target molecule (e.g., miRNA), providing a quantitative indicator (Step 6).

Alternatively, cleavage may selectively be performed on double-stranded oligonucleotides using a cleavage reagent specific to the duplex form. Using such a cleavage reagent would remove probes containing magnetic beads in proportion to the amount of bound target RNA, causing a decrease in signal due to loss of the magnetic beads. Since the probes have an attached label capable of modulating the capacitance measured by the capacitive touchscreen, a change in the number of probes at the conclusion of the assay changes the amount of label, thus modulating the signal.

An alternative to the differential measurement, or “delta” measurement previously described, wherein the capacitance is measured before and after application of the label and the difference is utilized as a signal, is to directly measure the capacitance of the label by taking a direct measurement of the signal, particularly in cases of very high signal-to-noise ratio where the signal will significantly dominate the noise.

Yet another alternative is to directly measure the signal from the probe site, and also directly measure the signal from another site not containing the probe, and taking the difference between the two measurements as the signal representing the capacitance generated by the probe.

The described biosensor may also be utilized to perform qualitative detection, for example binary detection, to determine the presence or absence of a target. Because the sensor system utilizes a capacitive sensing system rather than a fluorescent plate reader or other optical approach, the system is portable and does not require expensive instrumentation. In addition, the system may be utilized to provide real-time, or near real-time results.

Because commercial-off-the-shelf touchscreen hardware utilized in cellular telephones, smart phones, tablet computers, and other electronic devices has high capacitive sensitivity, such touchscreen components already embedded into such devices may be configured to perform the assay. One skilled in the art will appreciate that a standalone touchscreen separate from a cellular telephone, smart phone, tablet computer or other computing device is not required and such devices configured to provide the appropriate measurement may be utilized for the biosensing purposes described herein.

Modern touchscreens utilize low-cost highly sensitive capacitive sensor arrays to detect finger movements in the vicinity of the panel (with detection not requiring physical contact). Furthermore, such touchscreens increasingly incorporate multi-touch functionality, wherein the presence of multiple fingers can be detected at once. Such touchscreens may be utilized in assay development for examining DNA hybridization.

Multi-touch capable touchscreens may be adapted to simultaneously measure multiple probe sites, molecules, substances, targets, analytes, or other objects of interest. In one embodiment, unpackaged touchscreen development boards are utilized for use with oligonucleotide hybridization experiments along with software tools that provide low-level control over the touchscreen for the sensor readout. One skilled in the art will appreciate that gaining control over the touchscreen settings utilized to control or operate the embedded touchscreens allows the biosensor functionality described herein to be produced. Touchscreens generally utilize indium tin oxide (ITO) transparent metals films on glass substrates for their capacitive sensing. Transparent indium tin oxide electrodes are preferred as they allow labeled biomolecules that also contain a fluorescent label to be measured under a fluorescent microscope and the optical results to be correlated to the electrical measurements for characterization and validation purposes.

In order to leverage the cost benefits of economies of scale in the electronics industry, a preferred embodiment is to modify a high-volume commercial-off-the-shelf touchscreen for use in an assay, rather than to modify the components of the electronics hardware prior to assembly, for example, by developing a custom touchscreen not utilized in high-volume consumer applications. In one embodiment, labeled biomolecules can be placed directly in contact with the touchscreen surface. For example, it is preferable to deposit oligonucleotides onto the completed touchscreen surface, rather than to deposit the oligonucleotides onto a glass surface prior to assembly onto a touchscreen. Sealing of the panel along the edges is preferred in order to prevent liquids utilized in the assay from making contact with the electronics and potentially causing a short circuit that could interrupt proper electrical operation of the device. Exemplary encapsulants include RTV (room temperature vulcanizing silicone) and epoxy resins. Those skilled in the art often prefer to wash the surface of the glass prior to deposition of the oligonucleotide. This washing can involve the use of acid. Therefore, it is preferred that the device be capable of withstanding the harshness of acid. The device is intended to be disposable, and preferably single-use, so long-term resistance to harsh chemicals is not required.

Grounding of the sample on the touchscreen surface may be used, depending on the size and material properties of the object to be measured. It is important that the object to be measured resist changes to voltage in response to its position relative to the touchscreen. For example, the human finger, in contact with the human body, is effectively grounded such that a direct connection of the finger to the system ground may not be required. Other objects, e.g. a zinc penny, due to their smaller size or material properties, may be subject to charging, in which they take on the voltage of the touchscreen. By failing to maintain a voltage difference, the measurement of the target object is significantly diminished or even lost completely. Charging of the surface of the object, which negatively impacts the measurement, can be avoided by connecting the object to a system ground, for example, through a wire, integrated circuit, or printed circuit board trace.

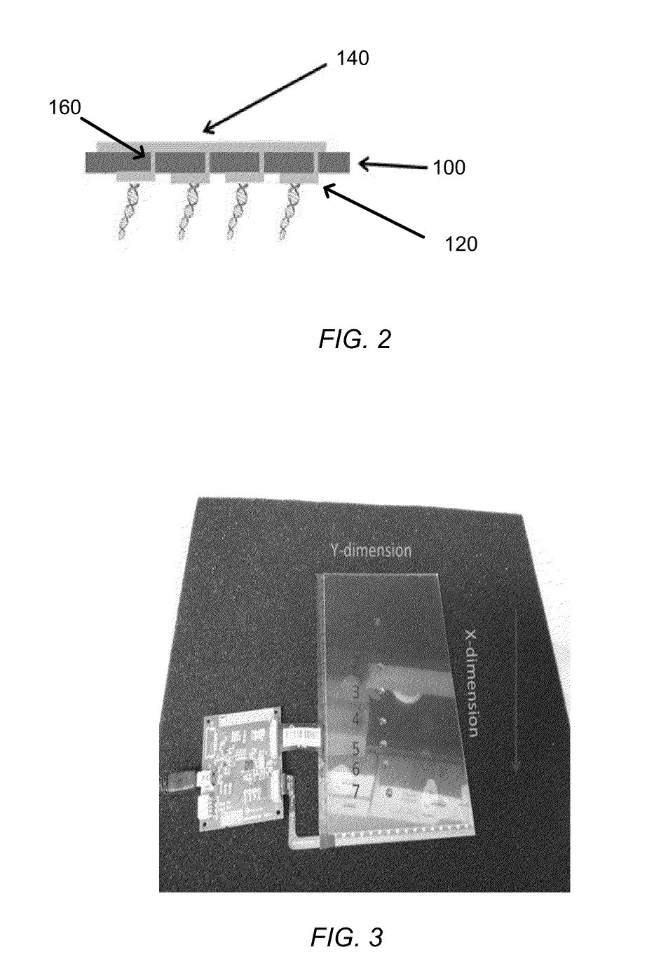

A second embodiment for performing an array of measurements of biological samples using a biosensor having an array or electrodes is illustrated in FIG. 2. Rather than directly deposit the probes onto the touchscreen surface, the probes are deposited onto a specially-designed printed circuit board 100. On one side of the printed circuit board, an array of electrodes 120 is established having conductive leads. The arrangement of the electrodes may be in a grid pattern. On the opposing side of the board, a large grounding layer or conductive trace 140 is established. Vias 160 connect electrodes 120 on the one side of the board to the grounding layer on the other side of the board so that they are in electrical contact. The grounding layer may be electrically connected to a system ground in the measurement system. Alternatively, if the grounding layer is substantial enough so as to allow the samples deposited on the electrode grid to avoid surface charging, connection of the grounding layer to the system ground may be unnecessary. The electrode grid of the printed circuit board is placed in contact with a capacitive touchscreen in order to perform the measurement. Such a configuration is advantageous since it enables projected capacitive touchscreens which are integrated into devices such as cellular phones, smart phones, or personal computers, or tablet computers, to be utilized as the reader. A further advantage is that the touchscreen is not required to be exposed to harsh chemicals utilized in any surface cleaning processes used in the deposition processes for the oligonucleotides. Adjusting the grounding has been found to have a significant impact on the ability to measure signals from labels contained on the touchscreen in a dry, solid-form, and can significantly enhance the signal-to-noise ratio.

Off-the-shelf projected capacitive touchscreen panels have numerous software layers and configuration settings tuning those devices to their primary input device, a human finger, and alternatively, a stylus. In order to produce the most suitable measurement, it is important to recognize that many of these software layers tuning the touchscreen to the characteristics of the human finger should be eliminated, adjusted, or adapted so as to enable the device to more appropriately measure the capacitance of the selected label. Examples of settings that should be altered include digitizer settings (interpreting a signaling event as a mouse click), anti-touch suppression, noise suppression, gain, touch threshold, touch hysteresis, touch detect integration, among others.

One skilled in the art should appreciate that any label sufficient to produce a measurable difference in capacitance in the presence of the touchscreen is a candidate for utilization as a label for the biological sample and incorporation into an assay.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

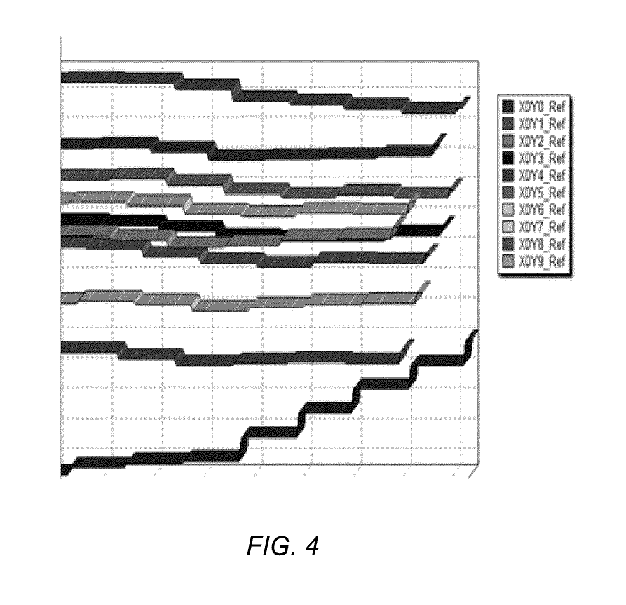

FIG. 3 depicts an experimental set up that includes a projected capacitive touchscreen coupled to a touchscreen controller. To test this methodology, water droplets were placed on the projected capacitive touchscreen. Referring to FIG. 4, water droplets were applied to a corner of the touchscreen at pixel location X0Y0. After application of several droplets, a rise in the capacitive signal was observed while other pixel locations in the line (X0Y1-X0Y9, serving as controls) remained stable.

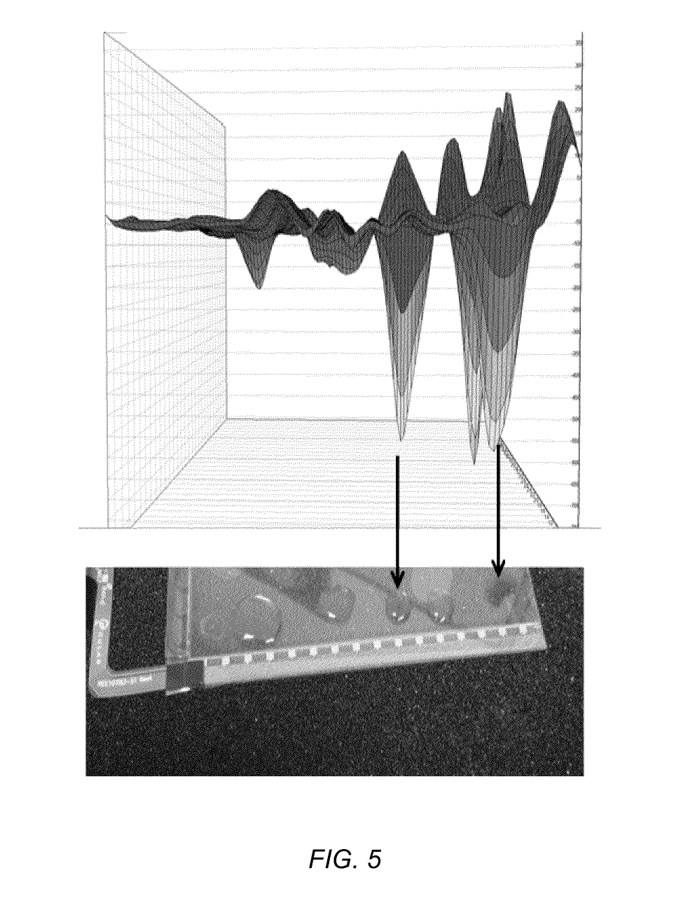

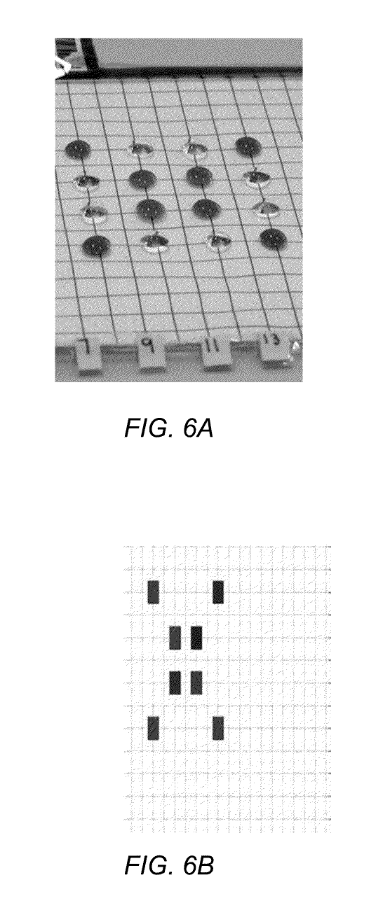

To amplify the water signal, droplets of magnetic beads were added to the surface, as shown in FIG. 5. The first two droplets (from left to right) included water only. The next three droplets were composed of water with magnetic beads. As shown, a much stronger signal was obtained using magnetic beads in the water. FIG. 6A depicts a projected capacitive touchscreen having water droplets and magnetic bead containing water droplets. FIG. 6B depicts the graphical output of the digital processing software which depicts the location of the water droplets that contain magnetic beads.

Based on this data, development of an assay device was undertaken comprising an array of magnetic bead-labeled single stranded probe DNAs which are immobilized on a solid support (as shown in FIG. 1. Immobilized magnetic bead-labeled single-stranded probe DNA is converted to double stranded form upon binding of complementary target nucleic acid (RNA or DNA), if present. Appropriate methods known to those skilled in the art can then be used to distinguish single stranded from double stranded probe. For example, nucleases able to specifically cleave single strand DNA but not double strand DNA can be used for this purpose. Cleavage of immobilized magnetic bead-labeled single-stranded probe DNA results in removal of the label from the specific position where the probe is immobilized on the solid support. The presence of target DNA is determined by comparing the amount of immobilized label present at a specific position on the array after treatment effective to remove single stranded DNA, with the amount of signal present after hybridization of the probe to a sample that may contain complementary target nucleic acid.

It is contemplated that this device and method will be especially useful for measuring levels of microRNA targets in biological samples. In essence, this approach would replace the expensive and bulky optical reader hardware conventionally used with fluorophores with low-cost touchscreen electronics to read out magnetic bead-based biomarkers or biomolecules bound to other appropriate labels. By binding the magnetic bead to the probe rather than the target, bias will be minimized.

A model system was created to demonstrate the feasibility of using the device and methods described herein for microRNA detection. miRNAs differentially expressed in breast cancer were selected for the biosensing experiments. The specific miRNA oligonucleotides explored include hsa-miR 125b-2-3p, hsa-miR 21-5p, and hsa-miR-200c-3p. A silicon wafer with gold pads was successfully fabricated for use with thiolated oligonucleotides due to the well-known binding of sulfur atoms to gold. The wafer was developed as a test platform for hybridization experiments due to the large number of bond pads that could be created through the batch fabrication and the potential to be utilized with liquid handling robotics to deposit a large number of miRNA probes on the surface. These Au bond pad arrays produce a checkerboard-like pattern when bound with fluorescently-labeled DNA.

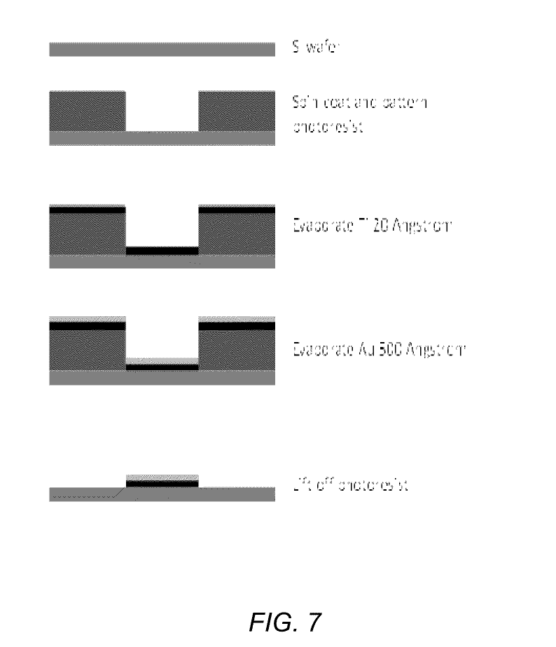

The fabrication process for this test platform it shown in FIG. 7. The device is fabricated using a silicon wafer with a single lithography lift-off process using metal evaporation with a Ti adhesion layer and Au bond pad layer. A lift-off process was successfully established using AZ 5209E positive photoresist with Nano-strip 2X (Cyantek, Fremont, Calif.) in place of acetone. Nano-strip was found to have a shorter soak time and to leave less residue compared to acetone, without etching the metallization.

In the present configuration, the 5′ end of the probe is bound to the touchscreen surface. The 3′end of the probe is linked via biotin/SA to the magnetic bead. Pre-synthesized oligonucleotides having modifications at the 5′ or 3′ end can be utilized. One skilled in the art will recognize that alternative configuration are also available, including binding the probe to the touchscreen surface using the 3′ end and linking the magnetic bead to the oligonucleotide at the 5′ end.

In order to perform experiments involving hybridization and as a means to evaluate shelf-life, a silicon chip with Au bond pads was designed to serve as a binding site for thiol-modified oligonucleotides. Two different pad sizes were developed including 75 μm and 150 μm gold bond pads. Fluorescently-labeled oligos immobilized on the gold bond pads can be examined under a fluorescent microscope. A 5″ photomask was designed and developed for use with 4″ silicon wafers. Using the cleanroom facilities, multiple wafers were successfully fabricated using the lift-off process shown in FIG. 7.

DNA binding experiments utilized miRNA oligonucleotides that are differentially expressed in breast cancer. Initial experiments utilized gold nanoparticles for attachment of the thiol group, while later experiments utilized either a sputtered Au/Pd alloy film or e-beam evaporated Au on Ti. Negative controls were successfully demonstrated with no signal being seen from buffer or unlabeled probe. A single base pair mismatch between probe and target showed decreased fluorescence as expected.

Deposition of the oligonucleotides onto the touchscreen surface can be performed using an arrayer, inkjet printing, or other suitable deposition means.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention will be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as examples of embodiments. Elements and materials may be substituted for those illustrated and described herein, parts and processes may be reversed, and certain features of the invention may be utilized independently, all as would be apparent to one skilled in the art after having the benefit of this description of the invention. Changes may be made in the elements described herein without departing from the spirit and scope of the invention as described in the following claims.

1-8. (canceled)

9. A biosensor comprising:
a substrate comprising a plurality of capacitive sensitive electrodes;
one or more single stranded probe oligonucleotides, wherein one end of each single stranded probe oligonucleotide is linked to a label and the other end of each single stranded probe oligonucleotide is coupled to the substrate proximate to one or the plurality of electrodes;
wherein the capacitance of the electrodes changes when the coupled single stranded probe oligonucleotide is removed or altered.

10. The biosensor of claim 9, wherein the substrate is a touchscreen.

11. The biosensor of claim 9, wherein the substrate is a capacitive touchscreen.

12. The biosensor of claim 9, wherein the substrate is a projected capacitive touchscreen.

13. The biosensor of claim 9, wherein the substrate comprises a glass layer formed over one or more of the electrodes, and wherein the one or more single stranded probe oligonucleotides are coupled to the glass layer.

14. The biosensor of claim 9, wherein one or more of the single stranded probe oligonucleotides are DNA oligonucleotides.

15. The biosensor of claim 9, wherein one or more of the single stranded probe oligonucleotides are capable of hybridizing with miRNA.

16. The biosensor of claim 9, wherein one or more of the single stranded probe oligonucleotides are capable of hybridizing with cDNA.

17. The biosensor of claim 9, wherein the substrate comprises glass having an electrode array formed of indium tin oxide on a surface of the glass.

18. The biosensor of claim 9, wherein one or more of the single stranded probe oligonucleotides have a magnetic bead attached to the 5′ end, wherein the magnetic bead is the label.

19. The biosensor of claim 9, wherein one or more of the single stranded probe oligonucleotides have a magnetic bead attached to the 3′ end, wherein the magnetic bead is the label.

20. The biosensor of claim 9, wherein one or more of the single stranded probe oligonucleotides are coupled to the substrate at the 3′ end.

21. The biosensor of claim 9, wherein one or more of the single stranded probe oligonucleotides are coupled to the substrate at the 5′ end.

22. The biosensor of claim 9, wherein the substrate is incorporated into a cell phone.

23-26. (canceled)

27. A biosensor comprising:
a first substrate comprising one or more electrodes on a first side of the substrate and a grounding layer on a second side of the substrate, the second side being opposite to the first side;
one or more single stranded probe oligonucleotides, wherein one end of each single stranded probe oligonucleotide is linked to a label and the other end of each single stranded probe oligonucleotide is coupled to the electrodes of the first substrate;
vias connecting the one or more electrodes on the first side of the first substrate to the grounding layer on the second side of the first substrate;
wherein the capacitance of the electrodes changes when the coupled single stranded probe oligonucleotide is removed or altered.

28. The biosensor of claim 27, where the grounding layer is placed in electrical contact with a ground.

29. The biosensor of claim 27, where the grounding layer is placed in electrical contact with a system ground.

30. The biosensor of claim 27, where the first substrate is a printed circuit board.

31. The biosensor of claim 27, where the first substrate is an integrated circuit.

32. The biosensor of claim 27, where the ground of the first substrate is in electrical contact with the system ground of the second substrate.

33-34. (canceled)
